(19) United States
(12) Patent Application Publication
(10) Pub. No.: US 2007/0157483 A1
(43) Pub. Date: Jul. 12, 2007

(54) Monocular PD ruler

(76) Inventor: David G. DuMais, Plano, TX (US)

Correspondence Address: MICHAEL L. DIAZ, P.C., 555 REPUBLIC DRIVE, SUITE 200, PIANO, TX, 75074, US

(21) Appl. No.: 11/328,212

(22) Filed: Jan. 10, 2006

(51) Int. Cl. A61B 5/103

(52) U.S. Cl. 33/512

(57) ABSTRACT

The present invention is a monocular pupillary distance (PD) ruler designed to help an eye care professional align the optical center of prescription eyewear with the patient's eye. The ruler is a planar body having an upper and a lower edge. A bridge is sized and shaped to fit upon the nose of a person being measured for eyeglasses. The bridge is located at a center point of the ruler. The ruler also includes units of measurements originating from the center point and measuring out from either side of the center point. The ruler may include a handhold extending from an end of the ruler. The handhold may be rotatable for ease of storage.

### FIELD OF THE INVENTION

This invention relates to optical measuring devices. Specifically, the present invention relates to a monocular pupillary distance (PD) ruler for use in fitting prescription eye glasses.

### DESCRIPTION OF THE RELATED ART

One of the most basic and important tools for the optician is the PD (pupillary distance) ruler. FIG. 1 is a front view of an existing PD ruler 10. The ruler is normally 150 millimeters long with graduated millimeters markings. A millimeter is the unit of measurement used in the optical field. The PD ruler can be used for measuring frame dimensions, as well as for measuring pupillary distance. Some PD rulers are made of plastic, while others are made of metal or wood.

The person's pupillary distance must be obtained to align the optical center of prescription glasses with the person's eye. The monocular PD measurement is the distance from the centerline of the nose to the center of each pupil. The PD measurement is always important but, when using high index materials, it becomes even more important to obtain a precise PD. For lenses having moderate to high power, greater precision of the PD is required. When fitting progressive lenses and aspheric lenses, an accurate monocular PD measurement is absolutely essential. First, PD measurements should always be monocular since the nose is seldom located centrally in the face.

However, existing PD rulers suffer from several disadvantages. First, the indicia 12 (FIG. 1) are typically arranged in sequential format from one end 14 to the opposite end 16. Thus, when an optician takes the monocular PD measurement of a person, the end of the ruler must be positioned in two different places on the person's face to measure from the center of the face to each pupil. In addition, when utilizing these existing PD rulers 10, it is oftentimes difficult for the optician to accurately determine the center of the face. Thus, the optician must correctly ascertain the center of the face by positioning the flat edge 18 perpendicular to the bridge of the person's nose. The center of the face is determined by an inaccurate placement of the flat edge 18 of the PD ruler on the bridge of the nose of the person. A measurement device is needed which easily and comfortably allows the optician to accurately measure monocular PD. It would be advantageous to have a measurement device which measures the monocular PD without requiring the repositioning of the PD ruler to measure the distances for both pupils. It is an object of the present invention to provide such an apparatus.

### SUMMARY OF THE INVENTION

In one aspect, the present invention is a measurement device for measuring monocular pupillary distance (PD) of a person for use in aligning an optical center of the lens of prescription eyeglasses with the person's eye. The measurement device includes a planar body having an upper edge and a lower edge. Additionally, indicia are located on a surface of the planar body. The indicia provide sequential units of distance measurement. The lower edge has a bridge located at a center point of the planar body. The bridge is sized to fit upon the nose of the person. The bridge is positioned on the nose of the person and the PD of each pupil is then measured.

In another aspect, the present invention is a PD ruler for use in measuring the monocular PD of a person. The ruler includes a planar body having an upper edge and a lower edge. Indicia are located on a surface of the planar body. The indicia provide sequential units of distance measurement, such as millimeters, which originates from a center point of the ruler and is sequentially numbered from the center point outward. The lower edge also has a bridge located at the center point of the planar body. The bridge is sized to fit upon the nose of the person. In addition, a handhold extends from an end of the planar body. The bridge is positioned on the nose of the person and the PD of each pupil is measured. The handhold may be rotatably affixed to the end to allow the handhold to rotate about a pivot point on the end of the planar body.

### BRIEF DESCRIPTION OF THE DRAWINGS

FIG. 1 is a front view of an existing PD ruler;

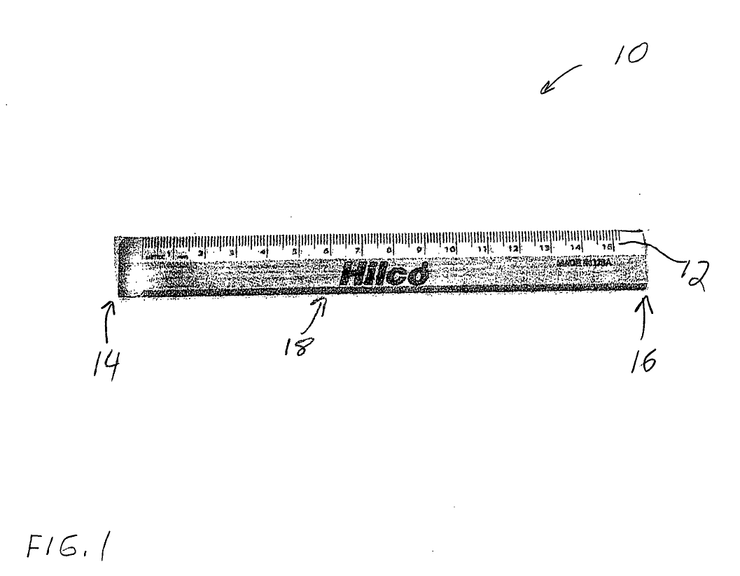

FIG. 2 is a front view of a PD ruler in the preferred embodiment of the present invention;

FIG. 3 is a front view of the PD ruler of FIG. 2 positioned upon the nose of a person; and

FIG. 4 is a front view of a PD ruler having a pivotable handhold in an alternate embodiment of the present invention.

### DESCRIPTION OF THE INVENTION

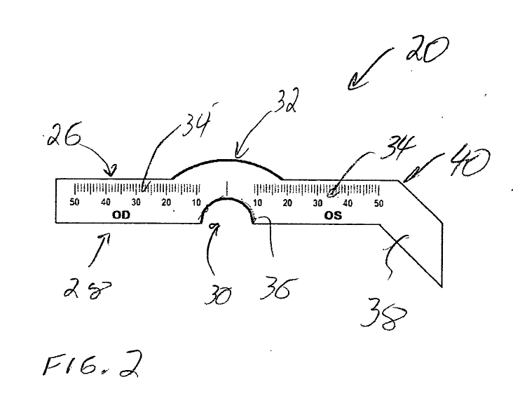

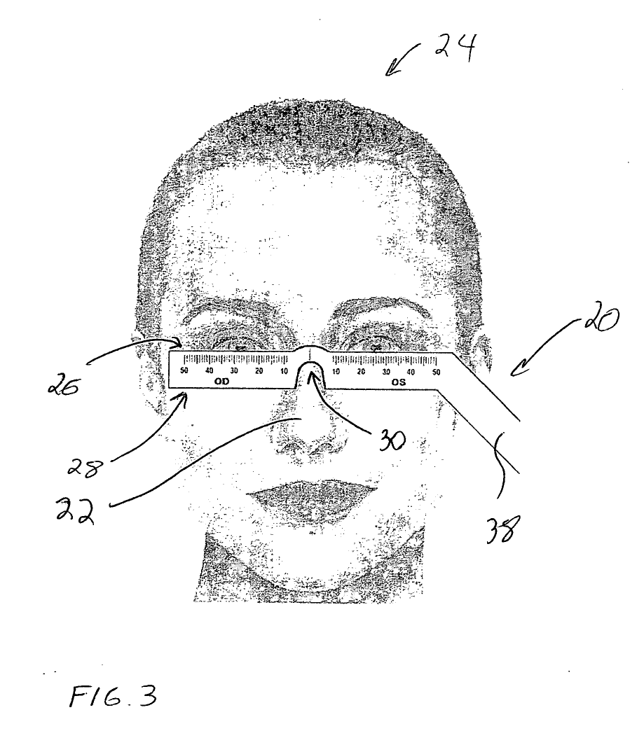

A monocular ruler for measuring the PD of a person utilized in fitting prescription eye glasses is disclosed. FIG. 2 is a front view of a PD ruler 20 in the preferred embodiment of the present invention. FIG. 3 is a front view of the PD ruler of FIG. 2 positioned upon the nose 22 of a person 24. The PD ruler includes an upper edge 26 and a lower edge 28. The lower edge includes a bridge 30 sized for positioning upon the nose of the person. The bridge is located at a center point 32 of the ruler. At the center point, indicia 34 are positioned at regular intervals outwardly from the center point. In the preferred embodiment of the present invention, the indicia are millimeter marks. Thus, from the center point, the marks run sequentially outward (e.g., 0-50 mm on the left side and 0-50 mm on the right side). The OD denotes placement over the person's right eye while the OS indicates the left eye.

The PD ruler 20 is constructed of a rigid material, such as wood, plastic or metal. The bridge may include a padded material 36 for added comfort for the person. Additionally, the PD ruler may include an optional handhold 38. The handhold may be positioned outwardly from an end 40 at an approximately 45 degree angle.

The PD ruler provides a monocular measurement which provides individual measurement of the distance between each pupil and the center of the person's nose. This measurement is used to align the optical center of prescription glasses with the person's eye. The use of a monocular ruler is critical because oftentimes a person's face is not symmetrical, thus measurement from the nose to each pupil is important for a proper fitting.

With reference to FIGS. 2 and 3, the operation of the PD ruler 20 will now be explained. The PD ruler is positioned on the person by placing the bridge 30 on the bridge of the nose 22 of the person in the same fashion that eyeglasses are positioned on a person's nose. This bridge holds the PD ruler 20 stationary while measurements are taken and also removes the guesswork in establishing the center of the nose. Thus, the optician may easily find the center of the person's face while providing a steady foundation for holding the PD ruler.

The PD ruler provides a unique measurement scheme whereby the indicia 34 includes sequentially numbered units measured from the center out. An existing ruler 10 measures in merely one direction. The existing ruler 10 requires measuring the binocular PD (from one eye to the other) and subtracting the distance to what is perceived as the center of the nose. Alternatively, the existing PD ruler must be centered on the nose and separate measurements are made outward to each pupil. The PD ruler 20, on the other hand, measures outward from the center point 32, thereby providing a far more accurate and rapid process in obtaining PD measurements.

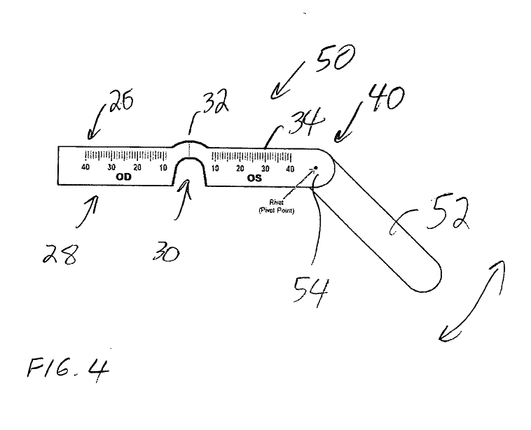

FIG. 4 is a front view of a PD ruler 50 having a pivotable handhold 52 in an alternate embodiment of the present invention. The PD ruler 50 is similar to the PD ruler 20 having an upper edge 26, a lower edge 28, a bridge 30, a center point 32, and indicia 34. However, the handhold 52 may be rotated about a pivot point 54 on the end 40 of the ruler. The handhold 52 may be rotatably affixed to the pivot point to allow the movement of the handhold 52. The PD ruler 50 allows the handhold 52 to be rotated to a positioned desired by the user (optician or other technician). Additionally, the handhold 52 may be folded up against the PD ruler for ease in storage.

The PD ruler may include different size bridges to accommodate persons of various sizes. The PD ruler may also include indicia in various types of units such as millimeters or inches.

The present invention provides many advantages over existing PD rulers. The present invention provides a bridge to allow steady placement of the PD ruler upon the nose of the person being measured. The present invention also allows the accurate and rapid capability to find the center of the person's face. The present invention utilizes novel measurements sequentially provided from the center outwardly to each end of the ruler to provide for rapid and easy monocular PD measurements. The present invention may also include a handhold which may be rotatable for compact storage.

While the present invention is described herein with reference to illustrative embodiments for particular applications, it should be understood that the invention is not limited thereto. Those having ordinary skill in the art and access to the teachings provided herein will recognize additional modifications, applications, and embodiments within the scope thereof and additional fields in which the present invention would be of significant utility.

Thus, the present invention has been described herein with reference to a particular embodiment for a particular application. Those having ordinary skill in the art and access to the present teachings will recognize additional modifications, applications and embodiments within the scope thereof.

It is therefore intended by the appended claims to cover any and all such applications, modifications and embodiments within the scope of the present invention.

What is claimed is:

1. A measurement device for measuring monocular pupillary distance (PD) of a person for use in aligning an optical center of the lens of prescription eyeglasses with the person's eye, the measurement device comprising:
a planar body having an upper edge and a lower edge;
indicia on a surface of the planar body, the indicia providing sequential units of distance measurement;
the lower edge having a bridge located at a center point of the planar body, the bridge sized to fit upon the nose of the person;
whereby the bridge is positioned on the nose of the person and the PD of each pupil is measured.

2. The measurement device for measuring monocular PD of claim 1 wherein in the indicia includes unitary distance measurements originating from the center point outward.

3. The measurement device for measuring monocular PD of claim 2 wherein the units of distance measurement are millimeters.

4. The measurement device for measuring monocular PD of claim 2 wherein the units of distance measurement are inches.

5. The measurement device for measuring monocular PD of claim 1 further comprising a handhold extending from an end of the planar body.

6. The measurement device for measuring monocular PD of claim 5 wherein the handhold extends from the end of the planar body at an approximately 45 degree angle.

7. The measurement device for measuring monocular PD of claim 5 wherein the handhold is rotatably affixed to the end to allow the handhold to rotate about a pivot point on the end of the planar body.

8. The measurement device for measuring monocular PD of claim 1 wherein the bridge is padded.

9. A measurement device for measuring monocular pupillary distance (PD) of a person for use in aligning an optical center of the lens of prescription eyeglasses with the person's eye, the measurement device comprising:
a planar body having an upper edge and a lower edge;
indicia on a surface of the planar body, the indicia providing sequential units of distance measurement, the indicia including units of measurements of millimeters originating and sequentially numbered from a center point of the planar body outward;
the lower edge having a bridge located at the center point of the planar body, the bridge sized to fit upon the nose of the person;
a handhold extending from an end of the planar body; whereby the bridge is positioned on the nose of the person and the PD of each pupil is measured.

10. The measurement device for measuring monocular PD of claim 9 wherein the handhold is rotatably affixed to the end to allow the handhold to rotate about a pivot point on the end of the planar body.
